(19) United States
(12) Patent Application Publication
(10) Pub. No.: US 2023/0295262 A1
(43) Pub. Date: Sep. 21, 2023

(54) PROGRAMMED CELLS FOR TARGETED ARTICULAR CARTILAGE AND BONE REGENERATION

(71) Applicants: Vanderbilt University, Nashville, TN (US); University of Tennessee Research Foundation, Knoxville, TN (US)

(72) Inventors: Jonathan M. Brunger, Nashville, TN (US); Karen A. Hasty, Knoxville, TN (US); Bonnie L. Walton, Nashville, TN (US); Craig L. Duvall, Nashville, TN (US)

(73) Assignees: Vanderbilt University, Nashville, TN (US); University of Tennessee Research Foundation, Knoxville, TN (US)

(21) Appl. No.: 18/120,191

(22) Filed: Mar. 10, 2023

Related U.S. Application Data
(60) Provisional application No. 63/320,567, filed on Mar. 16, 2022.
(60) Provisional application No. 63/320,907, filed on Mar. 17, 2022.

(51) Int. Cl. C07K 14/705; A61K 35/28; A61K 35/17; A61K 35/33; A61K 35/15; A61P 19/04; C12N 5/077; C12N 5/0775; C12N 9/16

(52) U.S. Cl. CPC C07K 14/705; A61K 35/28; A61K 35/17; A61K 35/33; A61K 35/15; A61P 19/04; C12N 5/0656; C12N 5/0663; C12N 9/16; C12Y 301/03001

(57) ABSTRACT

The disclosure describes engineered cells for the treatment of joint and bone disease. The cells are engineered to sense signals only found in diseased compartments and in turn produce therapeutic molecules that are secreted into the local environment.

### PRIORITY CLAIM

This application claims benefit of priority to U.S. Provisional Application Ser. No. 63/320,567, filed Mar. 16, 2022, and Ser. No. 63/320,907, filed Mar. 17, 2022, the entire contents of each application being hereby incorporated by reference.

### BACKGROUND

### I. Field

The present disclosure relates to the fields of molecular biology and medicine. More particularly, it relates to the fields of musculoskeletal tissues including cartilage and bone physiology, bone disease and injury, cartilage and bone repair.

### II. Related Art

Osteoarthritis (OA) affects over 32.5 million Americans, serves as the fifth leading cause of disability in older Americans, and carries an economic burden in excess of $139 billion per year. Various pathological mechanisms, ranging from mechanical trauma to dysregulated inflammatory cascades, contribute to the onset and progression of OA, while the defining feature of the disease remains cartilage deterioration. Although first-line treatments and lifestyle modifications serve to ameliorate pain and reduce progression of OA, no disease-modifying OA drugs (DMOADs) are available to restore joint homeostasis. Although targeted molecular therapies such as siRNA offer a great deal of promise toward the goal of reducing the burden of OA, limitations to these approaches include the need for repeat administration due to the half-life of drugs in the intra-articular space. As such, the progression of OA frequently leads to surgical procedures that include abrasion arthroplasty, the transplantation of autologous or allogeneic grafts, microfracture of the subchondral bone, or implantation of joint replacements. With the exception of the latter, a principal goal of these procedures is to encourage infiltration of progenitor or trophic cells to overcome the pathological hallmarks of OA and aid in the synthesis of a living tissue substitute that will restore a degree of joint function. Although these procedures offer some clinical benefit, outcomes decline after the initial post-operative period, primarily due to loss of appropriate chondrocyte phenotype in the degenerative environment, cell apoptosis, and a mismatch between the quality of tissue formed (i.e., fibrous rather than hyaline) and the physiologic demands placed upon it. This imposes a need to develop methods to support cellular regenerative functions that yield biomimetic tissue substitutes to restore articular homeostasis.

### SUMMARY

Thus, in accordance with the present disclosure, there is provided an engineered cell comprising a synthetic receptor functional via intramembrane proteolysis to generate a sense and response platform that controls expression of a therapeutic transgene. Features of the synthetic receptor include (1) an extracellular domain which serves as a recognition motif capable of engaging a target ligand; (2) a combination juxtamembrane/transmembrane domain which anchors the receptor in the cell membrane and contains protease cleavage sites that are exposed after receptor conformational changes take place in response to ligand binding; and (3) an intracellular domain that, upon ligand binding and subsequent protease-based cleavage of the receptor, is untethered from the receptor. Transmembrane domains can be selected from NOTCH4, SORT1, NOTCH1, PCDHGC3, APP, CLSTN1, NOTCH2, NOTCH3, CLSTN2, NECTIN1, and EPCAM. Juxtamembrane domains can be selected from NRG1, CSF1R, NOTCH3, NOTCH1, KCNE3, AGER, NOTCH4, PTPRF, PTPRM, NOTCH2, NRG2, LRP1B, PTPRF, JAG2, KL, EPHA4, PTPRK, CDH5, NOTCH1 and DAG1. In particular aspects, the sense and response platform may be synNotch, such as where a synNotch external domain is replaced with a heterologous recognition motif and the Notch intracellular domain is replaced with a selected transcription factor such as Gal4VP64 or the tetracycline transactivator. The sense and response platform maybe synNotch, such as where a Notch extracellular domain is replaced with a heterologous recognition motif and the Notch intracellular domain is replaced with a selected transcription factor such as Gal4VP64 or the tetracycline transactivator. The heterologous recognition motif may be an antigen binding domain, such as an scFv, a nanobody, or a high affinity peptide such as WYRGRL, or wherein the heterologous recognition motif recognizes collagen type II, collagen neo-epitopes, aggrecan neo-epitopes, or epitopes presented by damaged cartilage. The therapeutic transgene may be an antagonist of IL-1, IL-6, IL-17, TNF or complement. The transgene may be ILRa, TGF-β3, FGF2, IGF1, CXCL12, TIMP3, BMP4, sTNFR1, TGF-β1, IL-10, IL-4, SOX9, RUNX2, CRISPR-based transcriptome modulators (e.g., such as Rfx Cas13d, dCas9-VPR, dCas9-KRAB or CRISPRoff/DNMT3A-DNTM3L-dCas9-KRAB fusion protein or CRISPRon/TET1-XTEN80-dCas9 fusion protein). The engineered cell may comprise more than one therapeutic transgene. The engineered cell may be a mesenchymal stem cell (a.k.a. a multipotent mesenchymal stromal cell), a synovial fibroblast, a macrophage or a T cell. The cell may be engineered to produce neo-cartilaginous or neo-osseus tissues upon signal transduction via the synthetic receptor. Also provided is a pharmaceutical formulation comprising the engineered cell as defined herein.

In another embodiment, there is provided a method of increasing bone mass and/or volume and/or increasing cartilage mass and/or volume in a subject comprising (a) identifying a patient in need of increased bone mass and/or volume, and/or in need of increased cartilage mass and/or volume; and (b) administering to said subject an engineered cell as defined herein. The subject may be in need of increased bone mass and/or volume and/or in need of increased cartilage mass and/or volume. The engineered cell may be administered to said subject systemically. The engineered cell may be administered intravenously, intra-articularily, intra-peritoneally, intramuscularly, subcutaneously or topically. The engineered cell may be administered to a bone and/or cartilage target site, such as injected at said site. The engineered cell may be comprised in a time-release device implanted at said site.

The subject may be a human or a non-human animal, such as a mouse, a rat, a rabbit, a dog, a cat, a horse, a monkey or a cow. The subject may have cancer or may not have cancer. The human subject may be a subject of 60 years or older. The method may further comprise at least a second administration of said engineered cell, such as where the subject receives 1-5 administrations per week or receives at least 5 administrations. The method may further comprise assessing bone and/or cartilage mass and/or volume following administration of said engineered cell, such as by imaging. The subject may suffer from osteoarthritis, osteoporosis, bone fracture, spinal degeneration, alveolar/extraction socket defect, bone loss due to trauma, Paget's Disease or congenital bone diseases. The subject may suffer from bone loss due to cancer metastasis.

In yet another embodiment, there is provided a method of increasing bone and/or cartilage growth in a subject comprising administering to said subject an engineered cell as defined herein. The subject may be in need of increased bone growth and or in need of increased cartilage growth. The subject may have cancer or may not have cancer. The subject may be a human, such as a human subject of 60 years or older. The subject may be a non-human animal, such as a mouse, a rat, a rabbit, a dog, a cat, a horse, a monkey or a cow. The method may further comprise at least a second administration of said engineered cell, such as wherein said subject receives 1-5 administrations per week, or wherein said subject receives at least 5 administrations. The method may further comprise assessing bone and/or cartilage growth following administration of said engineered cell, such as by imaging.

The cells may be administered to the subject systemically, intravenously, intra-peritoneally, intramuscularly, subcutaneously, intraarticularly or topically. The cells may be administered to a bone target site, including injection at the site. The cells also may be comprised in a time-release device implanted at the site and may be a single entity or a combination of cells.

The non-human subject may be a human or a non-human animal, such as a mouse, a rat, a rabbit, a dog, a cat, a horse, a monkey or a cow. The subject may have cancer or may not.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Illustration of one implementation involving a modular synthetic Notch (synNotch) receptor (bottom left) whose detection motif is derived from the antibody mAbCII (bottom right, from Cho et al., 2018). This allowed creation of CII-synNotch, which enables cells to detect damaged cartilage and produce therapeutic transgenes in a disease-dependent manner. RK=right knee. LK=left knee. Further implementations involve use of recognition motifs that may enable other features of cartilage or bone damage, including recognition of neo-epitopes such as the NITEGE fragment from damaged cartilage matrix.

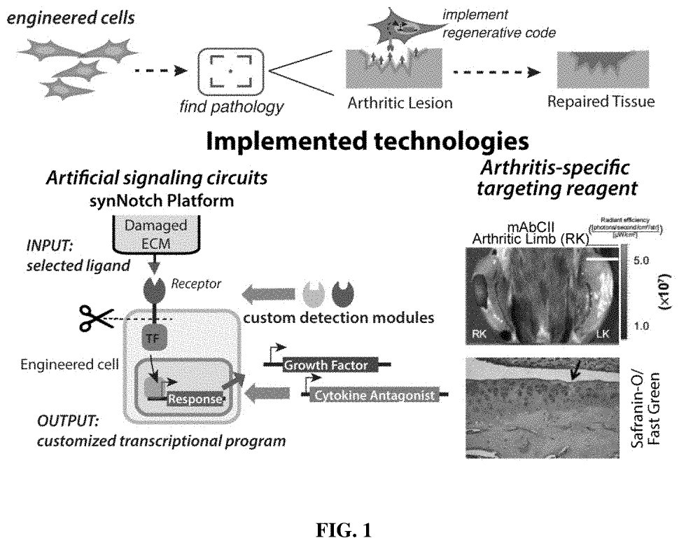

FIGS. 2A-C. CII-synNotch is sensitive and specific to CII inputs while potently inducing transgene expression. synNotch cells were cultured on surfaces treated with solutions of varied concentrations of type II collagen (CII). After 3 days, synNotch-induced luminescence was measured for fibroblasts (FIG. 2A) and murine MSCs (FIG. 2B). (FIG. 2C) Fibroblasts were cultured on either control, collagen I-treated dishes, or collagen II-treated surfaces. After 3 days, cells were lysed and luminescence was measured. **p<0.01, ****p<0.0001 by ANOVA followed with Tukey's post-hoc test.

FIGS. 3A-B. CII-synNotch accurately profiles cartilage degradation and spatially constrains transgene expression. (FIG. 3A) SEAP activity from MSCs seeded on healthy vs. TrypLE-treated porcine cartilage explants. TrypLE treatment exposes CII in the cartilage matrix leading to CII-synNotch activation. ***p<0.001, Student's t-test. (FIG. 3B) Explant (outlined in yellow dash) damaged via sectioning activates synNotch cells (red), whereas cells adjacent to the cartilage (blue) do not express the mCherry activation marker, highlighting the spatial gating provided by synNotch. Scale=200 mm.

FIG. 4. CII-synNotch tightly regulates expression of potentially therapeutic transgenes in mMSCs. ELISA showing CII-synNotch mMSC expression of either IL1Ra, sTNFR1, or TGFb3. In one group (green bar), the TGFb3 cell line was also treated with magnetic beads coated with anti-myc antibody, which can activate myc-tagged synNotch receptors as a positive control input to achieve maximal receptor activation. ****p<0.0001 by Student's t-test.

FIG. 5. CII-synNotch-driven IL1Ra expression protects chondrocytes from IL-1 signaling. Gene expression of ATDC5 cells in trans-wells with CII-synNotch mMSCs in ligand-free conditions or cultured on CII surfaces. Cultures were treated with 0 or 100 pg/ml IL-1, and after 3 days ATDC5 mRNA was collected for qRT-PCR. Groups not sharing the same letters are statistically significantly different (ANOVA, Tukey's post-hoc, p<0.05).

FIG. 6. Luminescence (left) and microscopy (center) establishing CII-synNotch performance in hMSCs. Scale=200 mm. **p<0.01 vs ctrl, Tukey HSD, n=3. (right) qRT-PCR analysis of IL1Ra expression induced by culture of engineered hMSCs on CII.

FIG. 7. Representative fluorescence microscopy of hMSCs seeded on one of 3 porcine cartilage explants. Image captured two weeks post-seeding. Scale=200 mm.

### DETAILED DESCRIPTION OF THE INVENTION

As discussed above, osteoarthritis is a significant health challenge. Current small molecules and biologics have limited benefit to treating osteoarthritis. Unlike such small molecule or biologic drugs, cells can integrate multiple inputs to perform an array of outputs as diverse as the assembly of neotissue or engulfment of foreign bodies. As such, cells can serve as exceptional agents to coordinate regeneration and combat disease. Wedding synthetic biology, the field that attempts to rationally assemble new biological systems that possess predictable and finely controlled properties, with cell therapies has resulted in remarkable outcomes in certain medical specialties. For example, the convergence of these domains resulted in the generation of chimeric antigen receptors (i.e., CARs) that allow oncologists to re-target T cells to eradicate previously intractable tumors.

The inventors envision treating complex musculoskeletal diseases such as OA with such living devices. Etiologies for OA include mechanical degeneration and biochemical factors brought about by injury, obesity, genetic disposition, and aging. Ultimately, these factors contribute to tissue catabolism mediated by direct tissue damage and/or chronic pro-inflammatory factors. Thus, their goal is to develop cells as sophisticated drug delivery vehicles that intelligently survey their microenvironment and activate spatiotemporally controlled functionalities “on demand” to combat inflammation and repair extracellular matrix (ECM) in the context of OA.

These and other aspects of the invention are set forth in detail below.

### I. ENGINEERED CELLS

Cell engineering strategies have been developed to overcome the limitations of surgical treatments. These strategies deploy cells or gene delivery vehicles to supply anabolic and/or anti-inflammatory factors to the joint; however, these rely on unregulated or only generically controlled expression of transgenes that may negatively impact health when expressed for long durations or outside of pathologic target tissues.

Here, the inventors propose to leverage synthetic biology tools to confine expression of transgenes to sites characterized by joint degeneration. Their approach builds on our use of a customized cell sense and response platform in regenerative engineering. This artificial cell signaling system enables one to program cells to react to selected features of a microenvironment by implementing defined transcriptional programs.

Specifically, first generation testing of this work involve use of a platform known as synNotch. SynNotch is based on the native juxtacrine Notch signaling channel, which requires mechanical strain generated by immobilized ligand for receptor activation. As such, synNotch activation is highly localized and depends on cell-ligand interactions within a niche; free soluble ligand does not activate this receptor. By exchanging Notch's (1) extracellular domain with alternative recognition motifs (i.e., scFVs or nanobodies) and (2) intracellular domain with a synthetic transcription factor, the inventors create synNotch receptors that produce user-specified sense/response behaviors. Since they can customize synNotch to drive expression of any transgene, they can tune defined cellular responses to selected microenvironmental inputs, opening the possibility to couple pathological microenvironmental cues to desired, rather than degenerative, cell phenotypes.

This disclosure capitalizes on the recent demonstration that the synthetic receptor platform selectively licenses cells to detect type II collagen and then upregulate expression of chosen transgenes. Completed experiments support that our programmed cells specifically recognize collagen II substrates, including collagen type II on degraded cartilage, and are capable of upregulating transgenes such as IL1Ra and TGFbeta-3 that may support cartilage matrix restoration. An ongoing goal is to establish synthetic receptor-controlled cells as agents to orchestrate cartilage repair and combat the inflammation associated with joint trauma or chronic joint inflammation.

### II. BONE AND CARTILAGE CONDITIONS

Bone and cartilage disease. There is a plethora of conditions which are characterized by the need to enhance bone formation or to inhibit bone resorption and thus would benefit from the use of engineered cells of the present disclosure, optionally further administered with agents, in promoting bone formation and/or bone repair. Perhaps the most obvious is the case of bone fractures, where it would be desirable to stimulate bone growth and to hasten and complete bone repair. Agents that enhance bone formation would also be useful in facial reconstruction procedures. Other bone deficit conditions include bone segmental defects, periodontal disease, metastatic bone disease, osteolytic bone disease and conditions where connective tissue repair would be beneficial, such as healing or regeneration of cartilage defects or injury. Also of great significance is the chronic condition of osteoporosis, including age-related osteoporosis and osteoporosis associated with post-menopausal hormone status. Other conditions characterized by the need for bone growth include primary and secondary hyperparathyroidism, disuse osteoporosis, diabetes-related osteoporosis, and glucocorticoid-related osteoporosis. Several other conditions, such as, for example, vitamin D deficiency, exists. There are also pediatric congenital conditions that are characterized by poor bone quality and amount including osteogenesis imperfecta that could benefit from enhanced bone formation and reduced bone turnover.

Osteoarthritis. Osteoarthritis (OA) is a type of degenerative joint disease that results from breakdown of joint cartilage and underlying bone. The most common symptoms are joint pain and stiffness. Usually, the symptoms progress slowly over years. Initially they may occur only after exercise but can become constant over time. Other symptoms may include joint swelling, decreased range of motion, and, when the back is affected, weakness or numbness of the arms and legs. The most commonly involved joints are the two near the ends of the fingers and the joint at the base of the thumbs; the knee and hip joints; and the joints of the neck and lower back. Joints on one side of the body are often more affected than those on the other. The symptoms can interfere with work and normal daily activities. Unlike some other types of arthritis, only the joints, not internal organs, are affected.

Causes include previous joint injury, abnormal joint or limb development, and inherited factors. Risk is greater in those who are overweight, have legs of different lengths, or have jobs that result in high levels of joint stress. Osteoarthritis is believed to be caused by mechanical stress on the joint and low grade inflammatory processes. It develops as cartilage is lost and the underlying bone becomes affected. As pain may make it difficult to exercise, muscle loss may occur. Diagnosis is typically based on signs and symptoms, with medical imaging and other tests used to support or rule out other problems. In contrast to rheumatoid arthritis, in osteoarthritis the joints do not become hot or red.

Treatment includes exercise, decreasing joint stress such as by rest or use of a cane, support groups, and pain medications.¹ Weight loss may help in those who are overweight. Pain medications may include paracetamol (acetaminophen) as well as NSAIDs such as naproxen or ibuprofen. Long-term opioid use is not recommended due to lack of information on benefits as well as risks of addiction and other side effects. Joint replacement surgery may be an option if there is ongoing disability despite other treatments. An artificial joint typically lasts 10 to 15 years.

The main symptom is pain, causing loss of ability and often stiffness. The pain is typically made worse by prolonged activity and relieved by rest. Stiffness is most common in the morning, and typically lasts less than thirty minutes after beginning daily activities, but may return after periods of inactivity. Osteoarthritis can cause a crackling noise (called “crepitus”) when the affected joint is moved, especially shoulder and knee joint. A person may also complain of joint locking and joint instability. These symptoms would affect their daily activities due to pain and stiffness. Some people report increased pain associated with cold temperature, high humidity, or a drop in barometric pressure, but studies have had mixed results.

Osteoarthritis commonly affects the hands, feet, spine, and the large weight-bearing joints, such as the hips and knees, although in theory, any joint in the body can be affected. As osteoarthritis progresses, movement patterns (such as gait), are typically affected. Osteoarthritis is the most common cause of a joint effusion of the knee.

In smaller joints, such as at the fingers, hard bony enlargements, called Heberden's nodes (on the distal interphalangeal joints) or Bouchard's nodes (on the proximal interphalangeal joints), may form, and though they are not necessarily painful, they do limit the movement of the fingers significantly. Osteoarthritis of the toes may be a factor causing formation of bunions, rendering them red or swollen.

Damage from mechanical stress with insufficient self-repair by joints is believed to be the primary cause of osteoarthritis. Sources of this stress may include misalignments of bones caused by congenital or pathogenic causes; mechanical injury; excess body weight; loss of strength in the muscles supporting a joint; and impairment of peripheral nerves, leading to sudden or uncoordinated movements. However, exercise, including running in the absence of injury, has not been found to increase the risk of knee osteoarthritis. Nor has cracking one's knuckles been found to play a role.

Rheumatoid arthritis. The exact etiology of RA remains unknown, but the first signs of joint disease appear in the synovial lining layer, with proliferation of synovial fibroblasts and their attachment to the articular surface at the joint margin. Subsequently, macrophages, T cells and other inflammatory cells are recruited into the joint, where they produce a number of mediators, including the cytokines interleukin-1 (IL-1), which contributes to the chronic sequelae leading to bone and cartilage destruction, and tumour necrosis factor (TNF-), which plays a role in inflammation. The concentration of IL-1 in plasma is significantly higher in patients with RA than in healthy individuals and, notably, plasma IL-1 levels correlate with RA disease activity. Moreover, synovial fluid levels of IL-1 are correlated with various radiographic and histologic features of RA.

In normal joints, the effects of these and other proinflammatory cytokines are balanced by a variety of anti-inflammatory cytokines and regulatory factors. The significance of this cytokine balance is illustrated in juvenile RA patients, who have cyclical increases in fever throughout the day. After each peak in fever, a factor that blocks the effects of IL-1 is found in serum and urine. This factor has been isolated, cloned and identified as IL-1 receptor antagonist (IL-1ra), a member of the IL-1 gene family. IL-1ra, as its name indicates, is a natural receptor antagonist that competes with IL-1 for binding to type I IL-1 receptors and, as a result, blocks the effects of IL-1. A 10- to 100-fold excess of IL-1ra may be needed to block IL-1 effectively, however, synovial cells isolated from patients with RA do not appear to produce enough IL-1ra to counteract the effects of IL-1.

Fracture. The first example is the otherwise healthy individual who suffers a fracture. Often, clinical bone fracture is treated by casting to alleviate pain and allow natural repair mechanisms to heal the wound. There has been progress in the treatment of fracture in recent times, however, even without considering the various complications that may arise in treating fractured bones, any new procedures to increase bone healing in normal circumstances would represent a great advance. There are a number of conditions in which bone fracture healing is defective, including metabolic diseases such as diabetes. Also, fracture healing is often poor in the elderly.

Periodontal Disease. Progressive periodontal disease leads to tooth loss through destruction of the tooth's attachment to the surrounding bone. Approximately 5-20% of the U.S. population (15-60 million individuals) suffers from severe generalized periodontal disease, and there are 2 million related surgical procedures. Moreover, if the disease is defined as the identification of at least one site of clinical attachment loss, then approximately 80% of all adults are affected, and 90% of those aged 55 to 64 years. If untreated, approximately 88% of affected individuals show moderate to rapid progression of the disease which shows a strong correlation with age. The major current treatment for periodontal disease is regenerative therapy consisting of replacement of lost periodontal tissues. The lost bone is usually treated with an individual's own bone and bone marrow, due to their high osteogenic potential. Bone allografts (between individuals) can also be performed using stored human bone. Although current periodontal cost analyses are hard to obtain, the size of the affected population and the current use of bone grafts as a first-order therapy strongly suggest that this area represents an attractive target for bone-building therapies.

Osteopenia/osteoporosis. The terms osteopenia and osteoporosis refers to a heterogeneous group of disorders characterized by decreased bone mass and fractures. Osteopenia is a bone mass that is one or more standard deviations below the mean bone mass for a population; osteoporosis is defined as 2.5 SD or lower. An estimated 20-25 million people are at increased risk for fracture because of site-specific bone loss. Risk factors for osteoporosis include increasing age, gender (more females), low bone mass, early menopause, race (Caucasians in general; Asian and Hispanic females), low calcium intake, reduced physical activity, genetic factors, environmental factors (including cigarette smoking and abuse of alcohol or caffeine), and deficiencies in neuromuscular control that create a propensity to fall.

More than a million fractures in the U.S. each year can be attributed to osteoporosis. In economic terms, the costs (exclusive of lost wages) for osteoporosis therapies are $35 billion worldwide. Demographic trends (i.e., the gradually increasing age of the U.S. population) suggest that these costs may increase to $62 billion by the year 2020. Clearly, osteoporosis is a significant health care problem.

Osteoporosis, once thought to be a natural part of aging among women, is no longer considered age or gender-dependent. Osteoporosis is defined as a skeletal disorder characterized by compromised bone strength predisposing to an increased risk of fracture. Bone strength reflects the integration of two main features: bone density and bone quality. Bone density is expressed as grams of mineral per area or volume and in any given individual is determined by peak bone mass and amount of bone loss. Bone quality refers to architecture, turnover, damage accumulation (e.g., microfractures) and mineralization. A fracture occurs when a failure-inducing force (e.g., trauma) is applied to osteoporotic bone.

Current therapies for osteoporosis patients focus on fracture prevention, not for promoting bone formation or fracture repair. This remains an important consideration because of the literature, which clearly states that significant morbidity and mortality are associated with prolonged bed rest in the elderly, particularly those who have suffered hip fractures. Complications of bed rest include blood clots and pneumonia. These complications are recognized and measures are usually taken to avoid them, but these is hardly the best approach to therapy. Thus, the osteoporotic patient population would benefit from new therapies designed to strengthen bone and speed up the fracture repair process, thus getting these people on their feet before the complications arise.

Bone Reconstruction/Grafting. A fourth example is related to bone reconstruction and, specifically, the ability to reconstruct defects in bone tissue that result from traumatic injury; as a consequence of cancer or cancer surgery; as a result of a birth defect; or as a result of aging. There is a significant need for more frequent orthopedic implants, and cranial and facial bone are particular targets for this type of reconstructive need. The availability of new implant materials, e.g., titanium, has permitted the repair of relatively large defects. Titanium implants provide excellent temporary stability across bony defects and are an excellent material for bone implants or artificial joints such as hip, knee and joint replacements. However, experience has shown that a lack of viable bone binding to implants the defect can result in exposure of the appliance to infection, structural instability and, ultimately, failure to repair the defect. Thus, a therapeutic agent that stimulates bone formation on or around the implant will facilitate more rapid recovery.

Autologous bone grafts are another possibility, but they have several demonstrated disadvantages in that they must be harvested from a donor site such as iliac crest or rib, they usually provide insufficient bone to completely fill the defect, and the bone that does form is sometimes prone to infection and resorption. Partially purified xenogeneic preparations are not practical for clinical use because microgram quantities are purified from kilograms of bovine bone, making large scale commercial production both costly and impractical. Allografts and demineralized bone preparations are therefore often employed, but suffer from their devitalized nature in that they only function as scaffolds for endogenous bone cell growth.

Microsurgical transfers of free bone grafts with attached soft tissue and blood vessels can close bony defects with an immediate source of blood supply to the graft. However, these techniques are time consuming, have been shown to produce a great deal of morbidity, and can only be used by specially trained individuals. Furthermore, the bone implant is often limited in quantity and is not readily contoured. In the mandible, for example, the majority of patients cannot wear dental appliances using presently accepted techniques (even after continuity is established), and thus gain little improvement in the ability to masticate.

In connection with bone reconstruction, specific problem areas for improvement are those concerned with treating large defects, such as created by trauma, birth defects, or particularly, following tumor resection; and also the area of artificial joints. The success of orthopaedic implants, interfaces and artificial joints could conceivably be improved if the surface of the implant, or a functional part of an implant, were to be coated with a bone stimulatory agent. The surface of implants could be coated with one or more appropriate materials in order to promote a more effective interaction with the biological site surrounding the implant and, ideally, to promote tissue repair.

Primary Bone Cancer and Metastatic Bone Disease. Bone cancer occurs infrequently while bone metastases are present in a wide range of cancers, including thyroid, kidney, and lung. Metastatic bone cancer is a chronic condition, survival from the time of diagnosis is variable depending on tumor type. In prostate and breast cancer and in multiple myeloma, survival time is measurable in years. For advanced lung cancer, it is measured in months. Cancer symptoms include pain, hypercalcemia, pathologic fracture, and spinal cord or nerve compression. Prognosis of metastatic bone cancer is influenced by primary tumor site, presence of extra-osseous disease, and the extent and tempo of the bone disease. Bone cancer/metastasis progression is determined by imaging tests and measurement of bone specific markers. Recent investigations show a strong correlation between the rate of bone resorption and clinical outcome, both in terms of disease progression or death.

Multiple Myeloma. Multiple myeloma (MM) is a B-lymphocyte malignancy characterized by the accumulation of malignant clonal plasma cells in the bone marrow. The clinical manifestations of the disease are due to the replacement of normal bone marrow components by abnormal plasma cells, with subsequent overproduction of a monoclonal immunoglobulin (M protein or M component), bone destruction, bone pain, anemia, hypercalcemia and renal dysfunction.

As distinct from other cancers that spread to the bone (e.g., breast, lung, thyroid, kidney, prostate), myeloma bone disease (MBD) is not a metastatic disease. Rather, myeloma cells are derived from the B-cells of the immune system that normally reside in the bone marrow and are therefore intimately associated with bone. Indeed, the bone marrow microenvironment plays an important role in the growth, survival and resistance to chemotherapy of the myeloma cells, which, in turn, regulate the increased bone loss associated with this disorder (world-wide-web at multiplemyeloma.org). Over 90% of myeloma patients have bone involvement, versus 40-60% of cancer patients who have bone metastasis, and over 80% have intractable bone pain. Additionally, approximately 30% of myeloma patients have hypercalcemia that is a result of the increased osteolytic activity associated with this disease (Cavo et al., 2006).

Common problems in myeloma are weakness, confusion and fatigue due to hypercalcemia. Headache, visual changes and retinopathy may be the result of hyperviscosity of the blood depending on the properties of the paraprotein. Finally, there may be radicular pain, loss of bowel or bladder control (due to involvement of spinal cord leading to cord compression) or carpal tunnel syndrome and other neuropathies (due to infiltration of peripheral nerves by amyloid). It may give rise to paraplegia in late presenting cases.

Myeloma Bone Disease. As discussed above, unlike the osteolysis associated with other bone tumors, the MBD lesions are unique in that they do not heal or repair, despite the patients' having many years of complete remission. Mechanistically, this seems to be related to the inhibition and/or loss of the bone-forming osteoblast during disease progression. Indeed, bone marker studies and histomorphometry indicate that both the bone-resorbing osteoclast and osteoblast activity are increased, but balanced early in the disease, whereas overt MBD shows high osteoclast activity and low osteoblast activity. Thus, MBD is a disorder in which bone formation and bone loss are uncoupled and would benefit from therapies that both stimulate bone formation and retard its loss.

A number of therapeutic approaches have been used in MBD, with the endpoints of treating pain, hypercalcemia, or the reduction of skeletal related events (SRE). Many of these may present serious complications. Surgery, such as vertebroplasty or kyphoplasty, that is performed for stability and pain relief has the attendant surgical risks (e.g., infection) made worse by a compromised immune system and does not reverse existing skeletal defects. Radiation therapy and radioisotope therapy are both used to prevent/control disease progression and have the typical risks of irradiation therapies. More recently, drugs such as the bisphosphonates that inhibit osteoclast activity have become a standard of therapy for MBD, despite the fact that they work poorly in this disorder. In 9 major double-blind, placebo-controlled trials on bisphosphonates, only 66% of patients showed an effective reduction in pain; 56% showed a reduction in SRE and only 1 of the 9 demonstrated a survival benefit.

Chondropathies. Several diseases can affect cartilage. Chondrodystrophies are a group of diseases, characterized by the disturbance of growth and subsequent ossification of cartilage. Some common diseases that affect the cartilage are listed below.

Osteoarthritis (discussed above) is a disease of the whole joint, however one of the most affected tissues is the articular cartilage. The cartilage covering bones (articular cartilage—a subset of hyaline cartilage) is thinned, eventually completely wearing away, resulting in a “bone against bone” within the joint, leading to reduced motion, and pain. Osteoarthritis affects the joints exposed to high stress and is therefore considered the result of “wear and tear” rather than a true disease. It is treated by arthroplasty, the replacement of the joint by a synthetic joint often made of a stainless-steel alloy (cobalt chromoly) and ultra-high molecular weight polyethylene (UHMWPE). Chondroitin sulfate or glucosamine sulfate supplements have been claimed to reduce the symptoms of osteoarthritis but there is little good evidence to support this claim.

Traumatic rupture or detachment is cartilage damage, often in the knee, and can be partially repaired through knee cartilage replacement therapy. Often when athletes talk of damaged “cartilage” in their knee, they are referring to a damaged meniscus (a fibrocartilage structure) and not the articular cartilage.

Achondroplasia results from reduced proliferation of chondrocytes in the epiphyseal plate of long bones during infancy and childhood, manifesting in dwarfism. Costochondritis is an inflammation of cartilage in the ribs, causing chest pain. Spinal disc herniation is an asymmetrical compression of an intervertebral disc ruptures the sac-like disc, causing a herniation of its soft content. The hernia often compresses the adjacent nerves and causes back pain. Relapsing polychondritis result from the destruction, probably autoimmune in nature, of cartilage, especially of the nose and ears, causing disfiguration. Death occurs by asphyxiation as the larynx loses its rigidity and collapses.

B. Combination Treatments

As discussed, the present invention provides for the treatment of bone disease and bone trauma by stimulating the production of new cartilage and bone tissue. Other agents may be used in combination with the engineered cells of the present disclosure. More generally, these agents would be provided in a combined amount (along with the cells) to produce any of the effects discussed above. This process may involve contacting the subject with both agents at the same time. This may be achieved by contacting the subject with a single composition or pharmacological formulation that includes both agents, or by contacting the cell or subject with two distinct compositions or formulations, at the same time, wherein one composition includes the cell and the other includes the second agent.

Alternatively, one agent may precede or follow the other by intervals ranging from minutes to weeks. In embodiments where the agents are applied separately to the cell or subject, one would generally ensure that a significant period of time did not expire between the time of each delivery, such that the agents would still be able to exert an advantageously combined effect on the cell or subject. In such instances, it is contemplated that one may contact the cell or subject with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, the engineered cell is “A” and the other agent is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration protocols and formulation of such agents will generally follow those of standard pharmaceutical drugs, as discussed further below. Combination agents include bisphosphonates (Didronel™, Fosamax™ and Actonel™), SERMs (Evista) or other hormone derivatives, and Parathyroid Hormone (PTH) analogs.

### III. PHARMACEUTICAL FORMULATIONS AND DELIVERY

A. Compositions and Routes

Pharmaceutical compositions of the present invention comprise an effective amount of one or more engineered cells dissolved or dispersed in a pharmaceutically acceptable carrier. The phrases “pharmaceutical or pharmacologically acceptable” refer to molecular entities and compositions that do not produce an adverse, allergic or other untoward reaction when administered to an animal, such as, for example, a human, as appropriate. The preparation of a pharmaceutical composition that contains at least one engineered cell, and optionally an additional active ingredient, will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, 1289-1329, incorporated herein by reference). Except insofar as any conventional carrier is incompatible with the active ingredient, its use in the pharmaceutical compositions is contemplated.

The engineered cell may be admixed with different types of carriers depending on how it is administered or is administered alone or in combination. The present invention can be administered intravenously, intradermally, transdermally, intrathecally, intraarterially, intraperitoneally, topically, intramuscularly, subcutaneously, topically, locally, injection, infusion, continuous infusion, localized perfusion bathing target cells directly, via a catheter in lipid compositions (e.g., nanoparticles, liposomes), or by other method or any combination of the forgoing as would be known to one of ordinary skill in the art (see, for example, Remington's Pharmaceutical Sciences, 18th Ed. Mack Printing Company, 1990, incorporated herein by reference). In particular, the engineered cell is formulated into a syringeable composition for use in intravenous administration.

The engineered cell may be formulated into a composition in a free base, neutral or salt form or ester. It may also be synthesized/formulated in a prodrug form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric, fumaric, or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include an engineered cell, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally-occurring or synthetic (i.e., designed or produced by man). Lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

One of ordinary skill in the art would be familiar with the range of techniques that can be employed for dispersing a composition in a lipid vehicle. For example, the BMP engineered cell may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

Naturally, the amount of the engineered cell in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose of engineered cell may also comprise from about 0.1 microgram/kg/body weight, about 0.2 microgram/kg/body weight, about 0.5 microgram/kg/body weight, about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In further embodiments, engineered cell may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,537,514, 6,613,308, 5,466,468, 5,543,158; 5,641,515; and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it may be desirable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sustained release formulations for treating of bone conditions include U.S. Pat. Nos. 4,722,948, 4,843,112, 4,975,526, 5,085,861, 5,162,114, 5,741,796 and 6,936,270, all of which are incorporated by reference. Methods and injectable compositions for bone repair are described in U.S. Pat. Nos. 4,863,732, 5,531,791, 5,840,290, 6,281,195, 6,288,043, 6,485,754, 6,662,805 and 7,008,433, all of which are incorporated by reference.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

B. Devices

In addition to providing cells for administration by routes discussed above, such agents, alone or in combination, maybe used in the context of devices, such as implants. A variety of bone related implants are contemplated, including dental implants, joint implants such as hips, knees, and elbows, vertebral/spinal implants, and others. The engineered cell may be disposed on a surface of the implant, including in a bioactive matrix or coating. The engineered cell may be further formulated to sustained, delayed, prolonged or time release. The coating may comprise polymers, for example, such as those listed below. The following is a list of U.S. patents relating to bone implants and devices which may be utilized in accordance with this embodiment of the invention:

TABLE A
BONE IMPLANT PATENTS

| U.S. Pat. Nos.* | Patent Title |
|---|---|
| 7,044,972 | Bone implant, in particular, an inter-vertebral implant |
| 7,022,137 | Bone hemi-lumbar interbody spinal fusion implant having an |
| | asymmetrical leading end and method of installation thereof |
| 7,001,551 | Method of forming a composite bone material implant |
| 6,994,726 | Dual function prosthetic bone implant and method for preparing the |
| | same |
| 6,989,031 | Hemi-interbody spinal implant manufactured from a major long bone |
| | ring or a bone composite |
| 6,988,015 | Bone implant |
| 6,981,975 | Method for inserting a spinal fusion implant having deployable bone |
| | engaging projections |
| 6,981,872 | Bone implant method of implanting, and kit for use in making |
| | implants, particularly useful with respect to dental implants |
| 6,929,662 | End member for a bone fusion implant |
| 6,923,830 | Spinal fusion implant having deployable bone engaging projections |
| 6,921,264 | Implant to be implanted in bone tissue or in bone tissue supplemented |
| | with bone substitute material |
| 6,918,766 | Method, arrangement and use of an implant for ensuring delivery of |
| | bioactive substance to the bone and/or tissue surrounding the implant |
| 6,913,621 | Flexible implant using partially demineralized bone |
| 6,899,734 | Modular implant for fusing adjacent bone structure |
| 6,860,884 | Implant for bone connector |
| 6,852,129 | Adjustable bone fusion implant and method |
| 6,802,845 | Implant for bone connector |
| 6,786,908 | Bone fracture support implant with non-metal spacers |
| 6,767,367 | Spinal fusion implant having deployable bone engaging projections |
| 6,761,738 | Reinforced molded implant formed of cortical bone |
| 6,755,832 | Bone plate implant |
| 6,730,129 | Implant for application in bone, method for producing such an |
| | implant, and use of such an implant |
| 6,689,167 | Method of using spinal fusion device, bone joining implant, and |
| | vertebral fusion implant |
| 6,689,136 | Implant for fixing two bone fragments to each other |
| 6,666,890 | Bone hemi-lumbar interbody spinal implant having an asymmetrical |
| | leading end and method of installation thereof |
| 6,652,592 | Segmentally demineralized bone implant |
| 6,648,917 | Adjustable bone fusion implant and method |
| 6,607,557 | Artificial bone graft implant |
| 6,599,322 | Method for producing undercut micro recesses in a surface, a surgical |
| | implant made thereby, and method for fixing an implant to bone |
| 6,562,074 | Adjustable bone fusion implant and method |
| 6,562,073 | Spinal bone implant |
| D473,944 | Bone implant |
| 6,540,770 | Reversible fixation device for securing an implant in bone |
| 6,537,277 | Implant for fixing a bone plate |
| 6,506,051 | Bone implant with intermediate member and expanding assembly |
| 6,478,825 | Implant, method of making same and use of the implant for the |
| | treatment of bone defects |
| 6,458,136 | Orthopaedic instrument for sizing implant sites and for pressurizing |
| | bone cement and a method for using the same |
| 6,447,545 | Self-aligning bone implant |
| 6,436,146 | Implant for treating ailments of a joint or a bone |
| 6,371,986 | Spinal fusion device, bone joining implant, and vertebral fusion |
| | implant |
| 6,370,418 | Device and method for measuring the position of a bone implant |
| 6,364,880 | Spinal implant with bone screws |
| 6,350,283 | Bone hemi-lumbar interbody spinal implant having an asymmetrical |
| | leading end and method of installation thereof |
| 6,350,126 | Bone implant |
| 6,287,343 | Threaded spinal implant with bone ingrowth openings |
| 6,270,346 | Dental implant for bone regrowth |
| 6,248,109 | Implant for interconnecting two bone fragments |
| 6,217,617 | Bone implant and method of securing |
| 6,214,050 | Expandable implant for inter-bone stabilization and adapted to |
| | extrude osteogenic material, and a method of stabilizing bones while |
| | extruding osteogenic material |
| 6,213,775 | Method of fastening an implant to a bone and an implant therefor |
| 6,206,923 | Flexible implant using partially demineralized bone |
| 6,203,545 | Implant for fixing bone fragments after an osteotomy |
| 6,149,689 | Implant as bone replacement |
| 6,149,688 | Artificial bone graft implant |
| 6,149,686 | Threaded spinal implant with bone ingrowth openings |
| 6,126,662 | Bone implant |
| 6,083,264 | Implant material for replacing or augmenting living bone tissue |
| | involving thermoplastic syntactic foam |
| 6,058,590 | Apparatus and methods for embedding a biocompatible material in a |
| | polymer bone implant |
| 6,018,094 | Implant and insert assembly for bone and uses thereof |
| 5,976,147 | Modular instrumentation for bone preparation and implant trial |
| | reduction of orthopedic implants |
| 5,906,488 | Releasable holding device preventing undesirable rotation during |
| | tightening of a screw connection in a bone anchored implant |
| 5,899,939 | Bone-derived implant for load-supporting applications |
| 5,895,425 | Bone implant |
| 5,890,902 | Implant bone locking mechanism and artificial periodontal ligament |
| | system |
| 5,885,287 | Self-tapping interbody bone implant |
| 5,819,748 | Implant for use in bone surgery |
| 5,810,589 | Dental implant abutment combination that reduces crestal bone |
| | stress |
| 5,759,035 | Bone fusion dental implant with hybrid anchor |
| 5,720,750 | Device for the preparation of a tubular bone for the insertion of an |
| | implant shaft |
| 5,709,683 | Interbody bone implant having conjoining stabilization features for |
| | bony fusion |
| 5,709,547 | Dental implant for anchorage in cortical bone |
| 5,674,725 | Implant materials having a phosphatase and an organophosphorus |
| | compound for in vivo mineralization of bone |
| 5,658,338 | Prosthetic modular bone fixation mantle and implant system |
| D381,080 | Combined metallic skull base surgical implant and bone flap |
| | fixation plate |
| 5,639,402 | Method for fabricating artificial bone implant green parts |
| 5,624,462 | Bone implant and method of securing |
| D378,314 | Bone spinal implant |
| 5,607,430 | Bone stabilization implant having a bone plate portion with integral |
| | cable clamping means |
| 5,571,185 | Process for the production of a bone implant and a bone implant |
| | produced thereby |
| 5,456,723 | Metallic implant anchorable to bone tissue for replacing a broken or |
| | diseased bone |
| 5,441,538 | Bone implant and method of securing |
| 5,405,388 | Bone biopsy implant |
| 5,397,358 | Bone implant |
| 5,383,935 | Prosthetic implant with self-generated current for early fixation in |
| | skeletal bone |
| 5,364,268 | Method for installing a dental implant fixture in cortical bone |
| 5,312,256 | Dental implant for vertical penetration, adapted to different degrees |
| | of hardness of the bone |

*The preceding patents are all hereby incorporated by reference in their entirety.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

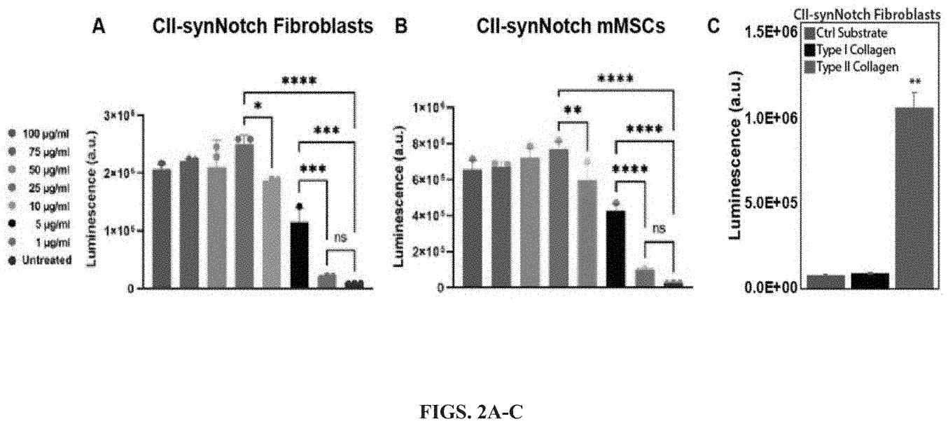

CII-synNotch, enables potent, programmable cell responses specific to type II collagen. Murine fibroblasts (L929 cells) and primary murine marrow-derived mesenchymal stem cells (mMSCs) were transduced with lentiviral vectors that carried synNotch platform components. In these experiments, synNotch activation results in simultaneous overexpression of the reporter transgenes firefly luciferase and mCherry. Culture surfaces were prepared as untreated cell culture surfaces, surfaces treated to passively adsorb type I collagen, or surfaces treated to passively adsorb type II collagen. Either fibroblasts or mMSCs were plated on these surfaces, and synNotch activation was assayed by quantifying firefly luminescence using a BrightGlo kit (Promega) on a plate reader. Results indicate that synNotch enables both fibroblasts and mMSCs to detect type II collagen adsorbed to culture dishes when CII solutions as low as 5 mg/ml are used for adsorption (FIGS. 2A-B). Further, at maximal activation using a solution of 25 mg/ml, synNotch cells were able to generate ˜35-fold induction of transgene expression over background levels. Finally, the inventors observed that this CII-synNotch receptor enables cells to distinguish between types I and II collagen, as type I collagen did not elicit any activation above background levels (FIG. 2C). These results confirm that CII-synNotch enables specific recognition of CII-substrates and potent inducible transgene expression.

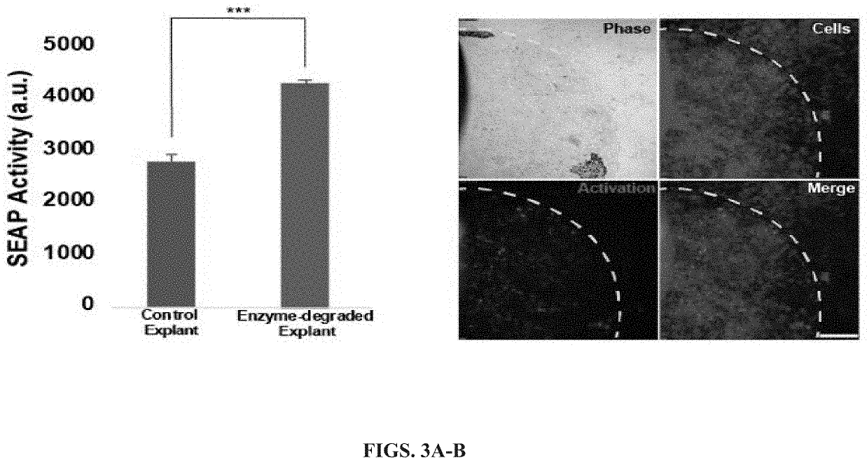

CII-synNotch cells accurately profile degraded vs. control cartilage explants. The inventors then transitioned from artificial, CII-decorated surfaces to more physiologically relevant substrates. They treated a subset of previously frozen porcine femoral cartilage explants with the dissociation reagent TrypLE Express for one hour to mimic the ECM degradation observed in severe arthropathy, while control explants were maintained in a solution of 10% FBS in PBS. After neutralizing the TrypLE with washes of 10% FBS solution, the inventors seeded mMSCs programmed with a CII-synNotch receptor that governed expression of the reporter transgene secreted alkaline phosphatase (SEAP) onto each explant. Culture medium from each sample was collected, and SEAP activity was measured using a luminescence assay (Takara). The inventors found increasing levels of SEAP activity in media collected from samples containing enzymatically damaged porcine cartilage, suggesting that, like mAbCII itself, CII-synNotch mMSCs can differentiate between healthy and damaged cartilage tissue (FIG. 3A).

Activation of CII-synNotch signaling is spatially constrained to damaged tissues. Porcine cartilage explants were cryosectioned to prepare 10 mm slices. CII-synNotch fibroblasts were then seeded on these physically damaged explants in cell culture. Fluorescent reporter transgene expression by CII-synNotch cells was monitored via microscopy. In these experiments, all synNotch cells constitutively express the blue fluorescent protein tagBFP and inducibly express mCherry upon synNotch activation. Fluorescence microscopy indicates that mCherry transgene expression is abundant in cells cultured on the cartilage cryosection, whereas cells out of contact with the cryosection are mCherry-low (FIG. 3B). Thus, CII-synNotch enables spatially regulated transgene expression, gated by cellular contact with damaged cartilage ECM.

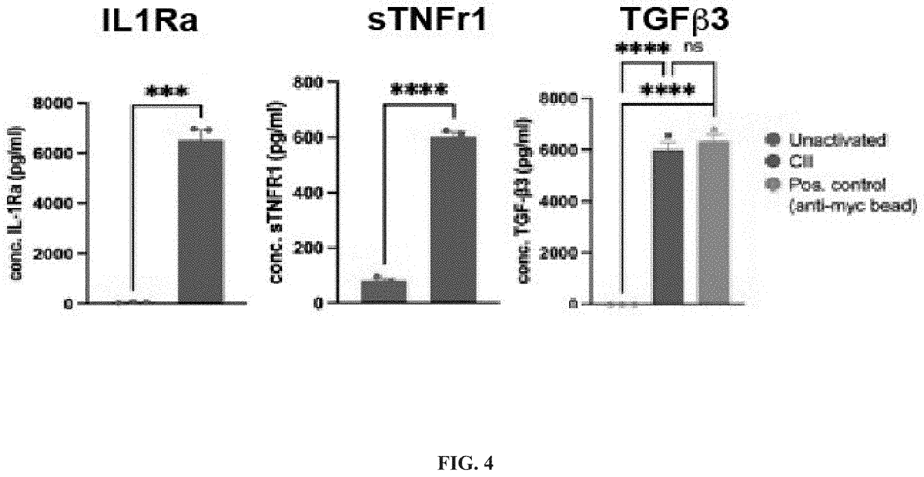

CII-synNotch cells implement cartilage regenerative functions in response to matrix degradation associated with OA. To determine whether CII-synNotch is capable of tightly regulating expression of potentially therapeutic transgenes from mMSCs in a ligand-dependent manner. Three CII-synNotch mMSC lines were generated: one to produce the IL-1 antagonist IL1Ra, one to produce the TNF antagonist sTNFR1, and a third to express the growth factor TGFb3. These cells were then cultured on control surfaces or surfaces treated with CII (25 mg/ml). The inventors collected media 3 days later to determine the production levels of these three factors via ELISA. They found dramatic and significant upregulation of all three of these transgene products (FIG. 4), highlighting the use of CII-synNotch for governing anti-inflammatory and anabolic transgene expression relevant to cartilage regenerative engineering.

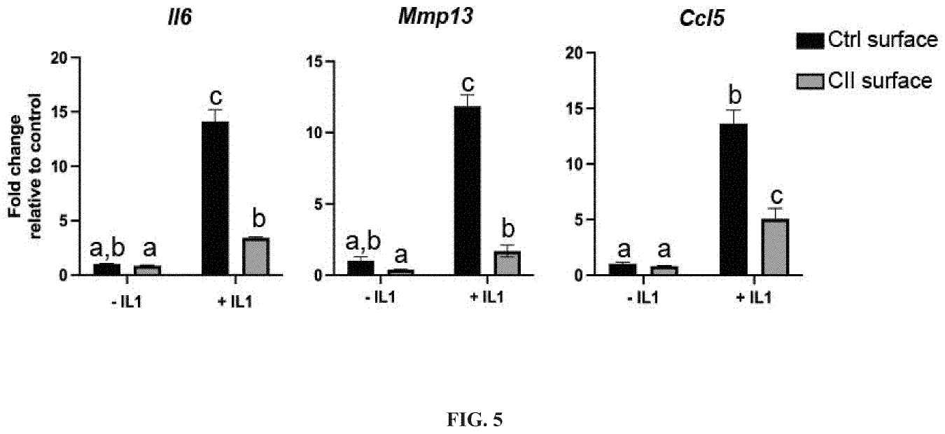

To determine whether the dramatic levels of expression measured in ELISA are of physiologic relevance, a trans-well co-culture experiment was performed. CII-synNotch mMSCs engineered to inducibly express IL1Ra were plated in the lower chamber of a trans-well on control or CII-treated surfaces, and ATDC5 chondrocytes were cultured on the trans-well mesh in the upper chamber. On the third day of culture, medium containing 0 or 100 pg/ml IL-1 was added to cultures. Three days later, RNA was isolated from ATDC5 cells, and qRT-PCR was performed to assay gene expression of a panel of transcripts known to be upregulated by IL-1 treatment of chondrocytes. Significant attenuation of the inflammatory transcripts 116, Ccl5 and Mmp13 in ATDC5 cells conditioned in the trans-wells with CII-activated synNotch MSCs was observed (FIG. 5). These data indicate that synNotch-mediated IL1Ra expression protected these chondrocytes from the deleterious impact of supraphysiologic IL-1 treatment.

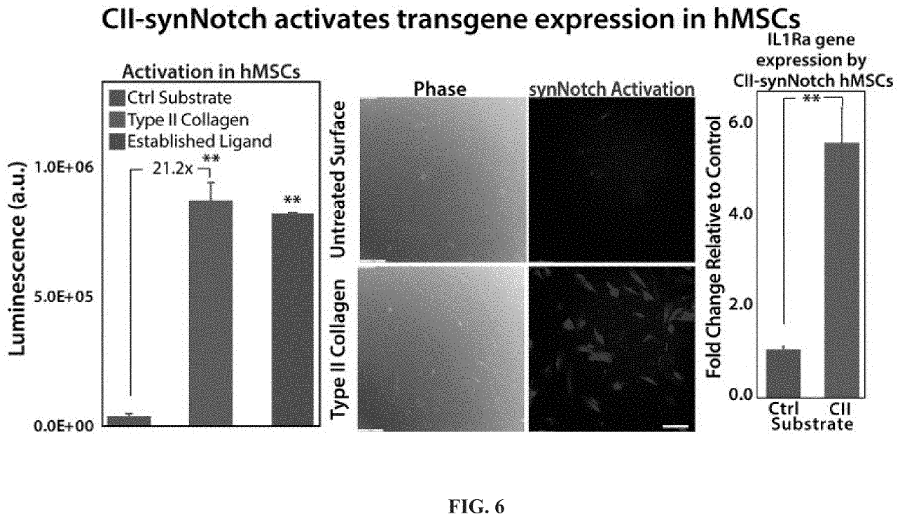

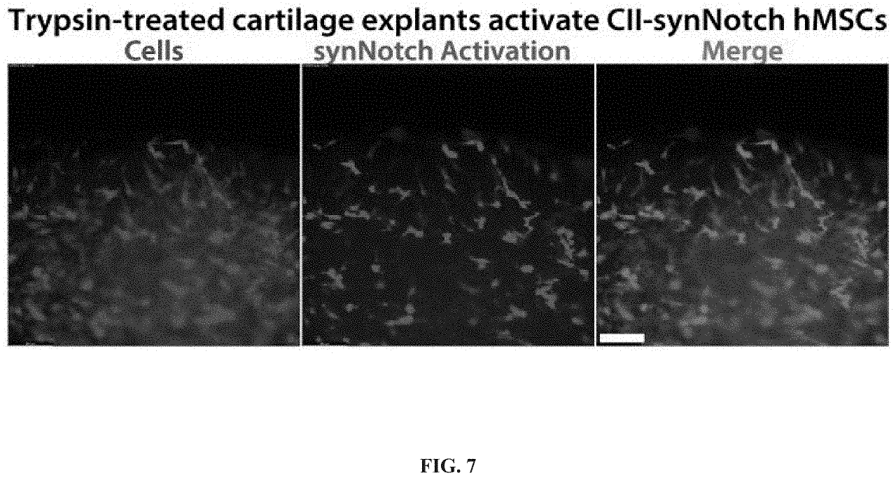

To translate these findings to more therapeutically relevant cells, subsequent experiments were performed with human MSCs (hMSCs). To test the basic function of CII-activated gene circuits in hMSCs, the inventors programmed cells to co-express luciferase and tagBFP in response to synNotch signaling. They then plated hMSCs on control or CII-coated polystyrene. After 6 days in culture, the inventors observed ˜21×induction of signaling in hMSCs in response to synNotch-mediated CII detection (FIGS. 6A-B). In a similar experiment, hMSCs engineered to express IL1Ra via synNotch upregulated expression 5.6-fold when cultured on CII as compared to untreated surfaces (FIG. 6C). Building on this, the inventors tested whether hMSCs seeded onto 6 mm porcine femoral cartilage explants (previously frozen, trypsin-treated [0.25% trypsin, 1 hr at 37° C.]) would upregulate transgene expression. The inventors engineered hMSCs to constitutively express BFP and inducibly express mCherry via synNotch. MCherry was strongly upregulated as detected by fluorescence microscopy (FIG. 7). These data provide direct evidence that the CU-detecting synNotch functions robustly in hMSCs and serves as a signaling channel whereby hMSCs recognize degraded articular cartilage tissue and respond as programmed.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An engineered cell comprising a synthetic receptor functional via intramembrane proteolysis to generate a sense and response platform that controls expression of a therapeutic transgene, said synthetic receptor comprising (1) an extracellular domain which serves as a recognition motif capable of engaging a target ligand; (2) a combination juxtamembrane/transmembrane domain which anchors the receptor in the cell membrane and contains protease cleavage sites that are exposed after receptor conformational changes take place in response to ligand binding; and (3) an intracellular domain that, upon ligand binding and subsequent protease-based cleavage of the receptor, is untethered from the receptor.

2. The engineered cell of claim 1, wherein the synthetic receptor comprises transmembrane domain, such as one selected from Notch4, SORT1, Notch1, PCDHGC3, APP, CLSTN1, NOTCH2, NOTCH3, CLSTN2, NECTIN1, and EPCAM, and a juxtamembrane domain, such as one selected from NRG1, CSF1R, NOTCH3, NOTCH1, KCNE3, AGER, NOTCH4, PTPRF, PTPRM, NOTCH2, NRG2, LRP1B, PTPRF, JAG2, KL, EPHA4, PTPRK, CDH5, NOTCH1 and DAG1.

3. The engineered cell of claim 1, wherein the sense and response platform is synNotch, such as where a Notch extracellular domain is replaced with a heterologous recognition motif and the Notch intracellular domain is replaced with a selected transcription factor such as Gal4VP64 or the tetracycline transactivator.

4. The engineered cell of claim 2, wherein the heterologous recognition motif is an antigen binding domain, such as an scFv, a nanobody, or a high affinity peptide such as WYRGRL, or wherein the heterologous recognition motif recognizes collagen type II, collagen neo-epitopes, aggrecan neo-epitopes, or epitopes presented by damaged cartilage.

5. The engineered cell of claim 1, wherein the therapeutic transgene is an antagonist of IL-1, IL-6, IL-17, TNF or complement.

6. The engineered cell of claim 1, wherein the therapeutic transgene is ILRa, TGF-β3, FGF2, IGF1, CXCL12, TIMP3, BMP4, sTNFR1, TGF-b1, IL-10, IL-4, SOX9, RUNX2, CRISPR-based transcriptome modulators (e.g. such as Rfx Cas13d, dCas9-VPR, dCas9-KRAB or CRISPRoff/DNMT3A-DNTM3L-dCas9-KRAB fusion protein or CRISPRon/TET1-XTEN80-dCas9 fusion protein).

7. The engineered cell of claim 1, wherein the engineered cell comprises more than one therapeutic transgene.

8. The engineered cell of claim 1, wherein the engineered cell is a mesenchymal stem cell (a.k.a. a multipotent mesenchymal stromal cell), a synovial fibroblast, a macrophage or a T cell.

9. The engineered cell of claim 1, wherein said cell is engineered to produce neo-cartilaginous or neo-osseus tissues upon signal transduction via the synthetic receptor.

10. A pharmaceutical formulation comprising the engineered cell of claim 1.

11. A method of increasing bone mass and/or volume and/or increasing cartilage mass and/or volume in a subject comprising:
(a) identifying a patient in need of increased bone mass and/or volume, and/or in need of increased cartilage mass and/or volume; and
(b) administering to said subject an engineered cell according to claim 1.

12. The method of claim 11, wherein the subject is in need of increased bone mass and/or volume.

13. The method of claim 11, wherein the subject is in need of increased cartilage mass and/or volume.

14. The method of claim 11, wherein said engineered cell is administered to said subject systemically.

15. (canceled)

16. The method of claim 1, wherein said engineered cell is administered to a bone and/or cartilage target site.

17. (canceled)

18. The method of claim 16, wherein said engineered cell is comprised in a time-release device implanted at said site.

19. The method of claim 11, wherein said subject is a human.

20. (canceled)

21. (canceled)

22. The method of claim 11, wherein said subject has cancer.

23. The method of claim 11, wherein said subject does not have cancer.

24. The method of claim 11, further comprising at least a second administration of said engineered cell.

25. (canceled)

26. (canceled)

27. The method of claim 11, further comprising assessing bone and/or cartilage mass and/or volume following administration of said engineered cell.

28. (canceled)

29. The method of claim 11, wherein said subject suffers from osteoarthritis, rheumatoid arthritis, osteoporosis, bone fracture, spinal degeneration, alveolar/extraction socket defect, bone loss due to trauma, Paget's Disease or congenital bone diseases, or bone loss due to cancer metastasis.

30. (canceled)

31. The method of claim 19, wherein said human subject is a subject of 60 years or older.

32. A method of increasing bone and/or cartilage growth in a subject comprising administering to said subject an engineered cell according to claim 1.

33. (canceled)

34. (canceled)

35. The method of claim 32, wherein said subject has cancer.

36. The method of claim 32, wherein said subject does not have cancer.

37. The method of claim 32, wherein said subject is a human.

38.-40. (canceled)

41. The method of claim 32, further comprising at least a second administration of said engineered cell.

42. (canceled)

43. (canceled)

44. The method of claim 32, further comprising assessing bone and/or cartilage growth following administration of said engineered cell.

45. (canceled)
